(19) United States
(12) Patent Application Publication
(10) Pub. No.: US 2019/0252074 A1
(43) Pub. Date: Aug. 15, 2019

(54) KNOWLEDGE GRAPH-BASED CLINICAL DIAGNOSIS ASSISTANT

(71) Applicant: KONINKLIJKE PHILIPS N.V., EINDHOVEN (NL)

(72) Inventors: Vivek Varma Datla, Ashland, MA (US); Sheikh Sadid Al Hasan, Cambridge, MA (US); Oladimeji Feyisetan Farri, Yorktown Heights, NY (US); Junyi Liu, Windham, NH (US); Kathy Mi Young Lee, Westford, MA (US); Ashequl Qadir, Melrose, MA (US); Adi Prakash, Waltham, MA (US)

(21) Appl. No.: 16/342,033

(22) PCT Filed: Oct. 24, 2017
(86) PCT No.: PCT/EP2017/077208

§ 371 Date: Apr. 15, 2019

Related U.S. Application Data
(60) Provisional application No. 62/533,400, filed on Jul. 17, 2017.
(60) Provisional application No. 62/412,329, filed on Oct. 25, 2016.

(51) Int. Cl. G16H 50/20; G16H 50/70; G06F 17/10; G06F 16/9032; G06F 16/907

(52) U.S. Cl. CPC G16H 50/20; G16H 50/70; G06F 16/907; G06F 16/90324; G06F 17/10

(57) ABSTRACT

A system (500) for automated clinical diagnosis includes: a knowledge graph (310, 510) generated using a curated corpus of medical information (520) and comprising a plurality of nodes; a user interface (512) configured to receive input comprising information about at least one patient symptom (316) and at least one patient demographic parameter (318); and a processor (530) configured to extract the at least one patient symptom and demographic parameter, and further configured to: (i) weight the extracted patient symptom; (ii) query the knowledge graph to generate a diagnosis graph as a subset of the knowledge graph; (iii) identify a ranked list of medical conditions for the patient from the diagnosis graph; and (iv) adjust, based on the extracted at least one demographic parameter about the patient, the ranking of the ranked list; wherein the identified medical conditions are provided to the user via the user interface.

### FIELD OF THE INVENTION

The present disclosure is directed generally to automated methods and systems to provide a clinical diagnosis of a patient's symptoms based on a corpus of medical knowledge.

### BACKGROUND

The diagnosis of a patient scenario is the hallmark of the clinician-patient interaction. Although some diagnoses are easy, many are often challenging for a clinician, as the clinician must perform complex cognitive processes to infer or hypothesize a diagnosis, determine which tests or tests to administer, and then determine a treatment in order to manage the medical condition(s) affecting the patient.

The standard of care for the diagnosis, testing, and treatment of patients performed by a clinician requires that the clinician have the most up-to-date knowledge available regarding the best management regimen across the entire care continuum. Ensuring up-to-date knowledge of many different fields can be extremely challenging. However, the cognitive burden of clinicians dealing with complex patient situations can be reduced or augmented by having an assistant who maintains not only current knowledge across the clinical and biomedical disciplines, but also recommends appropriate diagnoses and/or treatment for the patient in addition to the rationale for each option.

Existing systems or methods of automated clinical diagnosis of a patient situation are inadequate. For example, these existing systems do not update in real-time, and are unable to utilize natural language as an input option for the patient's scenario, among other limitations.

### SUMMARY OF THE INVENTION

There is a continued need for automated clinical diagnosis methods and systems that accept natural language input and that provide a diagnosis, testing plan, and/or treatment plan based on a corpus of medical knowledge updated in real-time.

The present disclosure is directed to inventive methods and systems for automated clinical diagnosis. Various embodiments and implementations herein are directed to a system that accepts natural language input from a medical professional about a patient's scenario. The system generates a digraph, which has symptoms as leaf nodes which are connected to diseases and medical conditions. The knowledge graph is updated in real-time taking into consideration information being generated in the digital universe of medical knowledge. The system processes the natural language input from the clinician and processes it with a natural language processing engine to extract the keywords related to symptoms, such as signs, lab results, procedures and demographic information. The symptoms are then processed over multiple cycles across the medical knowledge graph to generate a connected digraph that represents the connected symptoms. Activation and decay are propagated to obtain the maximal node weights which represent the symptoms and possible diagnoses. According to an embodiment, the possible diagnoses are tuned based on epidemiology to improve the accuracy of the recommendations relative to the patient scenario.

Generally, in one aspect, a system for automated clinical diagnosis is provided. The system includes: a knowledge graph generated from a corpus of medical information, the knowledge graph comprising a plurality of nodes, at least some of the nodes comprising a respective patient symptom and connected by an edge; a user interface configured to receive natural language input from a user, the input comprising information about at least one patient symptom and at least one demographic parameter about the patient; and a processor comprising a natural language processing engine configured to extract the at least one patient symptom and at least one demographic parameter from the received natural language input, wherein the processor is further configured to: (i) weight the extracted at least one patient symptom based at least in part on the frequency of the patient symptom in the corpus of medical information; (ii) query, using the weighted at least one patient symptom, the knowledge graph to generate a diagnosis graph as a subset of the knowledge graph; (iii) identify a ranked list of one or more medical conditions, diagnoses, treatments, and/or tests for the patient from the diagnosis graph; and (iv) adjusting, based on the extracted at least one demographic parameter about the patient, the ranking of the identified one or more medical conditions, diagnoses, treatments, and/or tests for the patient; wherein the identified one or more medical conditions, diagnoses, treatments, and/or tests for the patient are provided to the user via the user interface.

According to an embodiment, generating a diagnosis graph comprises the steps of: (i) assigning the assigned weight as an activation weight to a node of the knowledge graph; (ii) expanding the diagnosis graph to one or more connected nodes, wherein each expansion to a new connected node decays the activation weight; and (iii) concluding expansion when the activation weight is sufficiently decayed. According to an embodiment, the step of expanding the diagnosis graph to one or more connected nodes is repeated.

According to an embodiment, the processor comprises a control module configured to monitor the expansion and decay of the diagnosis graph. According to an embodiment, the control module is further configured to stop expansion of the diagnosis graph when the diagnosis graph stabilizes.

According to an embodiment, at least some of the edges of the knowledge graph are weighted.

According to an embodiment, the highest ranked one or more medical conditions, diagnoses, treatments, and/or tests for the patient is provided to the user.

According to an embodiment, the processor is further configured to: generate from the adjusted ranking of one or more medical conditions for the patient, a testing plan and/or treatment plan for the patient; and provide, to the clinician via the user interface, the generated testing plan and/or treatment plan for the patient.

According to an embodiment, the extracted at least one patient symptom is weighted based on the log inverse frequency of the symptom in the corpus of medical information.

According to an aspect is a method for automated clinical diagnosis. The method includes the steps of: (i) providing an automated clinical diagnosis system comprising a knowledge graph generated from a corpus of medical information, the knowledge graph comprising a plurality of nodes, at least some of the nodes comprising a respective patient symptom and connected by an edge; a user interface configured to receive input from a user, the input comprising information about at least one patient symptom and at least one demographic parameter about the patient; and a processor; (ii) receiving, via the user interface, information about a patient scenario, the information comprising at least one patient symptom and at least one demographic parameter for the patient; (iii) extracting, using the processor, the at least one patient symptom from the received information; (iv) extracting, using the processor, at least one demographic parameter for the patient from the received information; (v) weighting, using the processor, the extracted at least one patient symptom based at least in part on the frequency of the symptom in the curated corpus of medical information; (vi) querying, using the weighted at least one patient symptom, the knowledge graph to generate a diagnosis graph as a subset of the knowledge graph; (vii) identifying a ranked list of one or more medical conditions, diagnoses, treatments, and/or tests for the patient from the diagnosis graph; (viii) adjusting, based on the extracted at least one demographic parameter about the patient, the ranking of the identified one or more medical conditions, diagnoses, treatments, and/or tests for the patient; and (ix) providing the identified one or more medical conditions, diagnoses, treatments, and/or tests for the patient are provided to the user via the user interface.

According to an embodiment, the processor comprises a natural language processing engine configured to extract the at least one patient symptom and at least one demographic parameter from the received input.

According to an embodiment, the list of one or more medical conditions, diagnoses, treatments, and/or tests for the patient from the diagnosis graph is ranked based at least in part on information from one or more additional sources of medical information.

According to an embodiment, the step of querying the knowledge graph to generate a diagnosis graph as a subset of the knowledge graph comprises the steps of: assigning the assigned weight as an activation weight to a node of the knowledge graph; expanding the diagnosis graph to one or more connected nodes, wherein each expansion to a new connected node decays the activation weight; and concluding expansion when the activation weight is sufficiently decayed.

According to an embodiment, the method further includes the step of generating the knowledge graph from the corpus of medical information.

In various implementations, a processor or controller may be associated with one or more storage media (generically referred to herein as “memory,” e.g., volatile and non-volatile computer memory such as RAM, PROM, EPROM, and EEPROM, floppy disks, compact disks, optical disks, magnetic tape, etc.). In some implementations, the storage media may be encoded with one or more programs that, when executed on one or more processors and/or controllers, perform at least some of the functions discussed herein. Various storage media may be fixed within a processor or controller or may be transportable, such that the one or more programs stored thereon can be loaded into a processor or controller so as to implement various aspects of the present invention discussed herein. The terms “program” or “computer program” are used herein in a generic sense to refer to any type of computer code (e.g., software or microcode) that can be employed to program one or more processors or controllers.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

These and other aspects of the invention will be apparent from and elucidated with reference to the embodiment(s) described hereinafter.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the invention.

FIG. 1 is a flowchart of a method for automated clinical diagnosis, in accordance with an embodiment.

FIG. 2 is a flowchart of a method for automated clinical diagnosis, in accordance with an embodiment.

FIG. 3 is a schematic representation of a system for automated clinical diagnosis, in accordance with an embodiment.

FIG. 4 is a schematic representation of a system for automated clinical diagnosis, in accordance with an embodiment.

FIG. 5 is a schematic representation of a system for automated clinical diagnosis, in accordance with an embodiment.

### DETAILED DESCRIPTION OF EMBODIMENTS

The present disclosure describes various embodiments of an automated clinical diagnosis system. More generally, Applicant has recognized and appreciated that it would be beneficial to provide a system that accepts natural language input from a medical professional about a patient's scenario, processes the input, and provides one or more possible diagnoses, tests, and/or treatments. The system receives natural language input from a medical professional and processes the input using a natural language processing engine to extract the keywords related to symptoms, such as signs, lab results, procedures and demographic information. The system then analyses the symptoms over multiple cycles across the medical knowledge graph to generate a connected digraph that represents the connected symptoms. The results are summarized and provided to the clinician. According to an embodiment, the possible diagnoses are tuned based on epidemiology to improve the accuracy of the recommendations relative to the patient scenario.

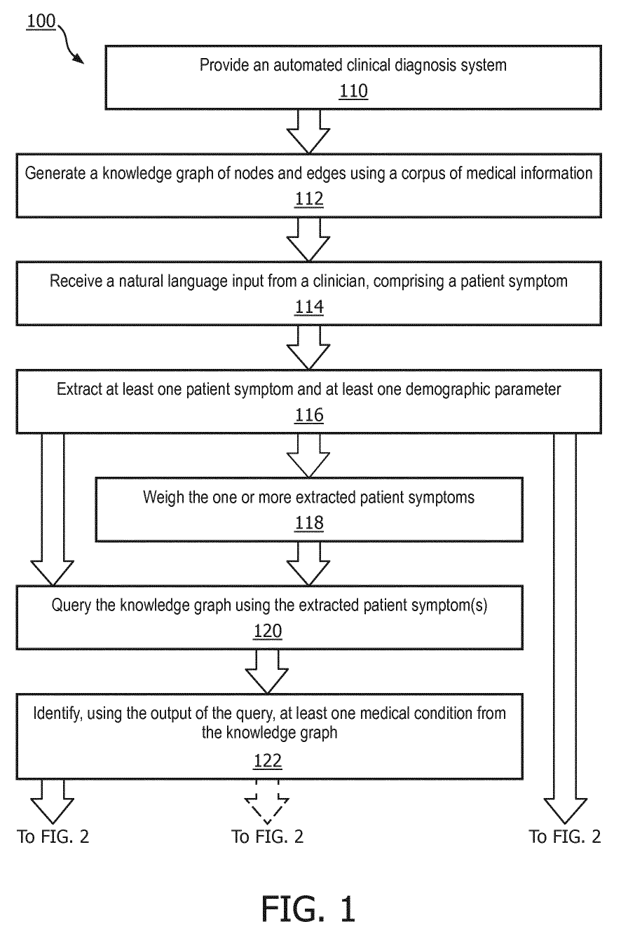

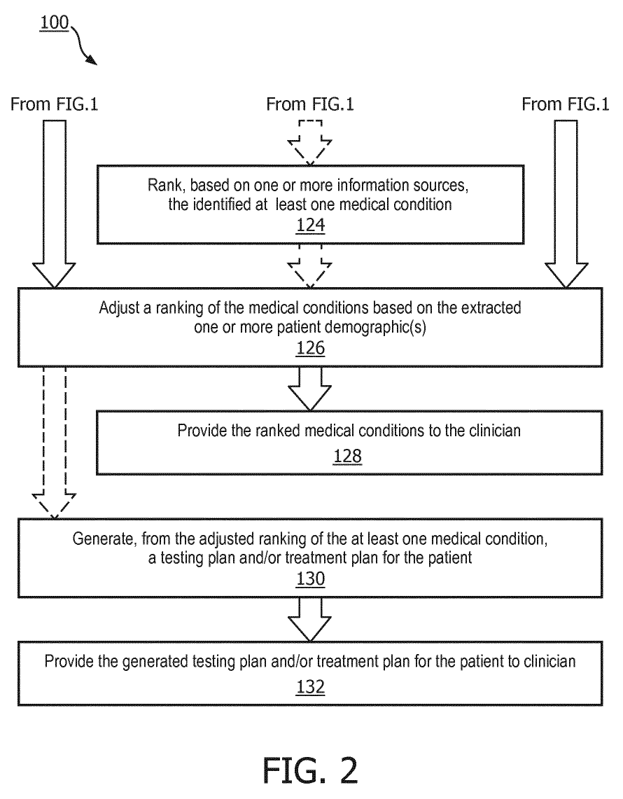

Referring to FIGS. 1 and 2, in one embodiment, is a flowchart of a method 100 for an automated clinical diagnosis system. At step 110 of the method, an automated clinical diagnosis system is provided. The clinical diagnosis system may be any of the systems described or otherwise envisioned herein.

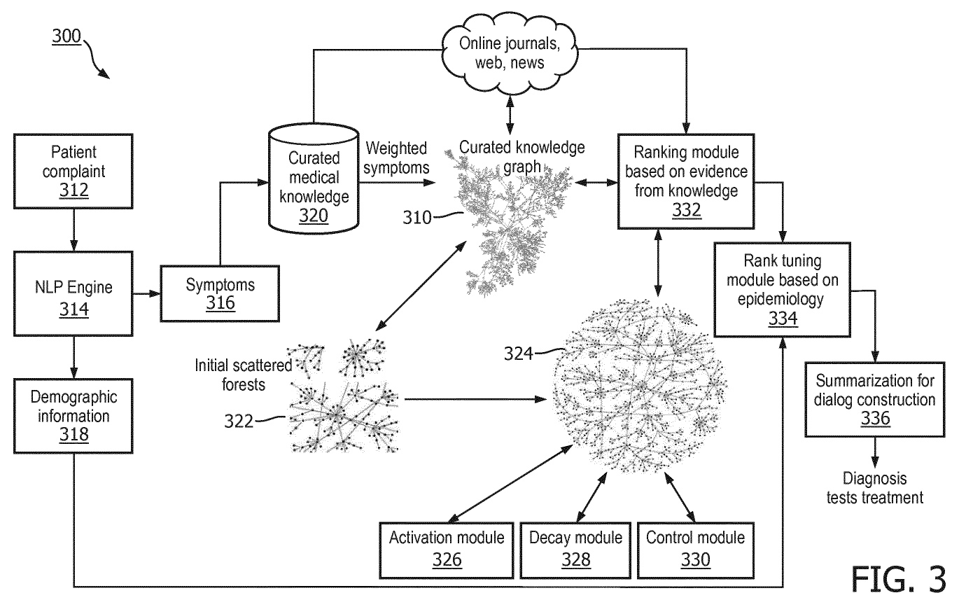

At step 112 of the method, a knowledge graph or diagraph 310 is constructed (as shown in FIG. 3). According to an embodiment, the knowledge graph is constructed from a corpus of medical information and comprises a plurality of interconnected nodes each comprising a different patient symptom. The corpus of medical information can be any source of information, including but not limited to medical journals, newspapers, online sources such as Wikipedia, and other sources. For example, when utilizing an online source with a hierarchy and a master top category (such as “clinical medicine”), all pages including and under the master top category are analyzed and the hierarchy of the sub-categorized pages is maintained, with information being extracted from each page. The information on one page or from one source may be inherently related to other pages, sources, or medical conditions, or these connections may be otherwise constructed or extracted. A directed graph is built using these interconnections and relations. For example, if a source or page has a link to another source or page, then the direction of the link is from the current source or page to the other source or page.

According to an embodiment, the knowledge graph 310 is a tree-like structure with a plurality of nodes connected by one or more edges. Each root node of the graph is a symptom, and the remaining nodes are conditions, diagnoses, tests, procedures, medications, or other clinical concepts. An edge is a relationship between two nodes. For example, a symptom of a fever will be connected by edges to hundreds of other nodes, as a fever is a symptom of many patient scenarios. As another example, a symptom of nystagmus will have few nodes as it is a symptom of fewer patient scenarios.

According to an embodiment, edges between the nodes are also weighted based on a relationship between the nodes. The relationship may be the frequency with which the two nodes are associated within a source or a corpus, or that the two nodes appear within the same source such as an online database or within the same medical journal, among other possible relationship systems. The weight may be variable and based on the strength of the relationship (for example, the frequency that the two nodes appear together) or other parameters of the nodes or their relationship. According to an embodiment, the graph is updated continuously and/or periodical as the medical knowledge source is updated. For example, the graph may be updated with new journal articles, new online sources, or other possible sources of new information.

According to an embodiment, the system retrieves a ranked list of the top 1000 biomedical articles that can answer generic clinical questions related to three categories: diagnosis, test, and treatment. Some embodiments may consider the importance of inferring the most probable clinical diagnosis from the given free text clinical scenario prior to biomedical article retrieval. Accordingly, various embodiments utilize a knowledge graph-based clinical diagnostic inference technique that can provide the most relevant diagnoses by analyzing the underlying context of the clinical narratives.

According to an embodiment, the system utilizes a graph-building approach that centers on three steps: (i) topical keyword analysis in which the most clinically relevant keywords from the given topic descriptions, summaries, and clinical notes are identified; (ii) diagnostic inference using reasoning based on the topical keywords to generate the diagnoses, tests, and treatments using the underlying clinical contexts represented within either a key-value memory network or a knowledge graph, both powered by an external clinical knowledge source; and/or (iii) relevant article retrieval in which pertinent biomedical articles are retrieved and/or ranked based on the topical keywords and clinical inferences from (i) and (ii) above.

Some embodiments use the Wiki pages under the clinical medicine category to build a knowledge graph. The hierarchy of each Wiki page is preserved to encode its distinguishing characteristics with respect to other pages. Each page consists of several sections and is related to other medical conditions. Some such embodiments build a directed graph (digraph) by using these relations, where each node is a medical condition, diagnosis, test, procedure, medication or any other clinical concept, and each edge is a relation between two nodes. If a page has a hyperlink to another page, then the direction of the edge is from the current page to the other page.

For example, the system may utilize Wikipedia clinical medicine category pages to build a directed knowledge graph 310, which possesses symptoms as root nodes connected by edges to the diseases and medical conditions associated with those symptoms. The knowledge graph is grounded as the activations flow directly from the root nodes to the entire graph. As described below, the grounded knowledge graph-based approach uses the activation-decay cycles to identify the most probable diagnosis given the description of the patient scenario in natural language.

In a next step of diagnostic inferencing, some embodiments use the extracted topical concepts from the previous step to infer relevant diagnoses, test, and/or treatment concepts from a clinical knowledge base derived from Wikipedia articles in the clinical medicine category, and embedded into a novel knowledge graph-based architecture. Some embodiments use a diagnostic inferencing approach where the system directly refers to the Wikipedia clinical knowledge base articles to extract a list of candidate articles with relevant diagnoses corresponding to each extracted topical keyword. Candidate Wikipedia articles can be filtered using various criteria e.g., location, gender, match with topical keywords etc., and the resulting list of Wikipedia articles with relevant clinical concepts can be mined to retrieve specific diagnoses (from the title of the Wikipedia article). Some embodiments alternatively build a novel end-to-end diagnostic inferencing model using Key-Value Memory Networks trained on a large col lection of MIMIC-II discharge notes along with the Wikipedia clinical knowledge base in order to capture the overall context of a given clinical note towards inferring the most probable diagnoses. Thereafter the list of possible diagnoses identified for all runs is used to extract a list of candidate Wikipedia articles to mine related tests, and treatments (from sections and subsections of the Wikipedia article) accordingly.

Key-Value Memory Networks (KV-MemNN) contain key-value paired memories, which uses a generalized approach of how the information is stored in memory. To solve question answering (QA) tasks, KV-MemNN first stores facts in key-value paired memory, uses the key to address relevant memories with respect to the question, and then extracts corresponding values. The addressing step takes place on the key memory and the reading step occurs on the value memory. The key is designed with features to help match it to the question (interest), while the value is designed with features to help match it to the final answer. According to an embodiment, the system adapts the KV-MemNN model to perform diagnostic inferencing from the given free text clinical narratives. Some embodiments extract knowledge for each diagnosis and store it to memory to help model infer the most probable diagnosis.

According to an embodiment, provided herein is one possible framework for collecting data, representing the data in the memory, and training the model. According to an embodiment, the system utilizes a MIMIC-II (Multiparameter Intelligent Monitoring in Intensive Care) dataset, which contains physiologic signals and vital signs in a time series format captured from patient monitors, and comprehensive clinical data obtained from hospital medical information systems, for tens of thousands of Intensive Care Unit patients. Some embodiments use the MIMIC-II discharge notes, which generally contain comprehensive clinical scenarios represented as unstructured free texts. Some such embodiments separate diagnosis from each medical record to create a collection of <medical note, diagnosis> pairs from this dataset. Then, some embodiments collect knowledge for each diagnosis from the Wikipedia pages under the clinical medicine category. Some diagnoses only have few instances in the data set. Without enough training instances, the model may not be able to learn to recognize these diagnoses. Hence, some embodiments only select the most common diagnoses with frequency value >50 yielding to 71 diagnoses for 8K medical note instances and thus, formulate the clinical diagnostic inferencing task as a multiclassmultilabel classification problem.

According to an embodiment, Wikipedia is a reasonable source for medical domain knowledge, since WikiProject Medicine is dedicated to improving the quality of medical articles in Wikipedia. Since certain diagnosis terms from MIMIC-II do not exactly match the Wikipedia page titles, some embodiments use the Wikipedia API to search for the most appropriate Wiki page by using each diagnosis term as the search keyword. According to an embodiment, the title of each Wikipedia page is the name of the diagnosis described by the page. The first section of such a Wiki page normally contains an introduction to the diagnosis. Among several other sections inside the Wiki page, the “Signs and symptoms” section describes the classic and common signs and symptoms for the diagnosis. Each collected Wikipedia page is turned into a key-value pair by using the following principle: the free text from the first section and the sections for sign and symptoms is the key and the title of the page is the value.

Similar to the KVMemNNs model, in some embodiments of the clinical diagnostic inferencing task the memory slots are defined as pairs of vectors (k1; v1); (k2; v2); (km; vm), where m is the size of memory, and clinical notes from MIMIC-II are denoted as x. The addressing and reading of the memory involve three steps:

Step one is key address. According to an embodiment, each memory slot is associated with a probability by measuring the similarity between the medical note and each key:

pₕ_{ᵢ}=Softmax(AΦ_{X}(x)·AΦ_{K}(kₕ_{ᵢ})) (Eq. 1)

where Φ are the feature maps of dimension D, and A denotes a d×D matrix. The softmax function is computed as: Softmax=exp (zi)=P j exp(zj). The medical note n is represented by A ΦX(x).

Step two is value reading. According to an embodiment, the reading output vectors o are computed by taking a weighted sum of the memory values based on the probabilities calculated at the previous step:

$$o = {\sum }_{i} {ph}_{i} A Φ ( {v}_{{h}_{i}} ) \qquad ( Eq . 2 )$$

Step three is note updating. According to an embodiment, after calculating o, the medical note is updated with the following equation:

nᵢ₊₁=Rᵢ(nᵢ+o) (Eq. 3)

where R denotes a d×d matrix.

These three steps are repeated with a different matrix Rᵢ in each hop. After a fixed number of H hops, the final probability for each diagnosis is computed using the final result o over all possible diagnoses:

\hat{p}=sigmoid*n_{H+1}^{T}BΦ_{Y}(yᵢ) (Eq. 4)

where yi represents a possible diagnosis and B is ad×D matrix.

The model is trained in an end-to-end fashion. Backpropagation and stochastic gradient descent algorithms are used to learn the parameters A, B and R₁; . . . ; R_{H}. Various embodiments use a simple bag-of-word (BoW) representation that transfers each word wᵢⱼ in the document di=wᵢ₁; wᵢ₂; wᵢ₃; . . . ; wᵢₙ to corresponding vector embeddings and sums these together to the resulting vectors: Φ(di)=Σⱼ Awᵢⱼ, where A denotes the embedding matrix.

As the next (in some embodiments, final) step, topical keywords and the corresponding diagnoses, tests, and treatments obtained from the diagnostic inferencing step can be used to retrieve candidate biomedical articles by searching through the given TREC-CDS corpus of over 1.25M PubMed Central articles (indexed using Elasticsearch). The retrieved candidate articles can be ranked using multiple weighting algorithms specific to the three types of clinical questions (diagnosis, test, and treatment). The biomedical articles can be further filtered by location (e.g. USA/Canada), demographic information and other contextual information from the topic description, summary or note towards improving the relevance of the results. The final list of top 1000 biomedical articles can be ordered by article publication date to provide chronological biomedical evidence for the answers to each topic.

In some embodiments, the test dataset comprises 30 topics divided into three question types: topic 1-10 (diagnosis), topic 11-20 (test), and topic 21-30 (treatment). The given topics are essentially medical case narratives that describe scenarios related to patient's medical history, signs/symptoms, diagnoses, tests, and treatments. The topics are provided in three versions depending on the depth of information. Besides topic “descriptions” that include comprehensive descriptions of the patient's situation and topic “summaries” that contain an abridged version of the most important information, topic “notes” are introduced this year, which are actual admission notes derived from MIMIC-III containing numerous abbreviations and domain-specific jargons. For example, some embodiments use a snapshot of the open access portion of PubMed Central (PMC), a freely available online database of full-text biomedical articles comprising 1.25M biomedical publications.

In some embodiments, a KV-MemNN model may be implemented using a TensorFlow framework. Such embodiments may use Adam stochastic gradient descent for optimizing the learned parameters. The learning rate may be set, for example, to 0:005 and the batch size for each iteration may be set to 100. As the final prediction layer, some embodiments may use a fully connected layer on top of the output layer from Eq. 4. The model may learn the parameters by minimizing a standard cross-entropy loss between a predicted diagnosis and the correct diagnosis. For regularization, some embodiments may use dropout with the probability 0:5 at the end of each hop and limit the norm of the gradients to below 4. Some embodiments may train the model on 80% of the data for 200 epochs using batch gradient descent while the remaining 20% data was equally divided to a validation and a testing set. All hyperparameters may be chosen based on the model's performance on validation data. Finally, the learned model may be used to predict the most probable diagnoses from the given medical notes for each topic.

At step 114 of the method, a clinician, medical professional, or patient provides information to the automated system via a user interface. The information is provided in natural language, and contains information about at least one patient symptom and at least one demographic parameter for the patient. For example, the information may be provided using any method or system, or any source. For example, the question may be received from a user in real-time, such as from a mobile device, laptop, desktop, wearable device, home computing device, or any other computing device. The question may be received from any user interface that allows information to be received, such as a microphone or text input, among many other types of user interfaces.

The at least one patient symptom may be any symptom or condition, whether normal, abnormal, or otherwise. For example, the patient symptom may be fever, flushing, sweating, and/or any other known patient condition or symptom. The at least one demographic parameter for the patient may be any demographic information about the patient. For example, the demographic information may be age, height, weight, medical background, sex, or any of a wide variety of other demographic information.

At step 116 of the method, a natural language processing engine, module, or system analyzes the information provided via the user interface. The natural language processing engine extracts at least one patient symptom from the received information and at least one demographic parameter for the patient from the received information. For example, the natural language processing engine may extract keywords related to symptoms, such as lab results, procedures, and/or demographic information.

At step 118 of the method, the extracted one or more patient symptoms are weighted by the system based at least in part on the frequency of the symptom in the curated corpus of medical information. According to an embodiment, the symptoms are weighted based on specificity of their usage using log inverse frequency of their usage in the medical corpus utilized to generate the knowledge graph. Other methods of weighting the patient symptoms are possible.

According to an embodiment, the system may extract term frequency-inverse document frequency (TFIDF) weighted topical keywords from the given descriptions, summaries or notes and map them to categories represented in one or more ontologies, including but not limited to the following controlled clinical ontologies: SNOMED CT for diagnoses, LOINC for tests, and/or RxNorm for treatments. Furthermore, various embodiments may identify relevant demographic information, interpret vital signs based on standard normal range values, and/or filter out negated clinical concepts in order to give more weight to positive clinical manifestations in a given clinical scenario.

At step 120 of the method, the system queries the knowledge using the extracted one or more patient symptoms, which may or may not be weighted. According to an embodiment, querying the knowledge graph using the weighted one or more patient symptoms comprises generating a diagnosis graph subset of the knowledge graph. Generating a diagnosis graph subset of the knowledge graph may comprise one or more of: (i) assigning, to a node of the knowledge graph comprising the extracted one or more patient symptoms, the assigned weight as an activation weight; (ii) expanding to one or more connected nodes, wherein each expansion to a new connected node decays the activation weight; and (iii) concluding expansion when the activation weight is sufficiently decayed.

According to an embodiment, the system begins a new forest starting with the root nodes in the knowledge graph and expanding nodes outward until a spanning tree is created, and an activation-decay cycle applied to the spanning tree, the medical conditions/diseases are ranked. According to an embodiment, the symptoms are processed over multiple cycles across the knowledge graph to generate a connected digraph that represents the connected symptoms. Activation and decay are propagated to obtain the maximal node weights which represent the symptoms and possible diagnoses.

According to an embodiment, the knowledge graph is grounded as the activations flow directly from the root nodes to the entire graph. The grounded digraph-based approach exploits the activation-decay cycles to identify the most probable diagnosis given a clinical narrative, such as a summary, description, or note. When the TF-IDF weighted clinical concepts extracted from the clinical narrative are used to query the knowledge graph, some embodiments perform all one-hop expansion of the symptom nodes towards building a digraph with the activation weights initialized to the associated TF-IDF weights. The nodes of the initial scattered forests having the least number of children are then expanded such that a connected graph is formed. This expansion is based on a minimal context addition principle, where the objective is to build a connected digraph by minimizing the number of nodes. The expansion is discontinued when a spanning tree structure is found or created. The activation module spreads the activation across the digraph and is controlled using a sigmoid function. Only partial activation flows to its children as inheritance of activation is proportional to number of siblings of the current node. Activation is a continuous process and it spreads from parent to children across the nodes in the same fashion. As the activation spreads concurrently, various embodiments decay the activation. Each time during the inheritance of activation the nodes lose a variable amount of activation based on the distance of a node from the initial node. Therefore, the nodes that are farther away from the base receive the most decayed activation.

According to an embodiment, the system comprises a control module that monitors the activation and decay cycle, and ensures that there is no runaway activation among the nodes. This module also controls the accumulation of activations at each node and stops the activation and decay cycle when the network is stabilized.

At step 122 of the method, at least one medical condition and/or diagnosis is identified from the knowledge graph based at least in part on the output of the query in step 160. For example, once the network is stable, one or more top-ranked diseases and medical conditions can be extracted from the knowledge graph. According to an embodiment, the identified one or more medical conditions and/or diagnosis can be ranked based in part on information from one or more additional sources of medical information.

At optional step 124 of the method, the identified at least one medical condition and/or diagnosis is ranked based in part on information from one or more additional sources of medical information. For example, signs and symptom information from online sources and curated medical sources can be used to rank or refine the list of diseases and medical conditions.

At step 126 of the method, a ranking of the identified at least one medical condition and/or diagnosis is adjusted based on the extracted at least one demographic parameter for the patient. Accordingly, the demographic information obtained from the clinical narrative is leveraged to fine-tune the ranking. For example, if a disease is not common for a demographic, its rank is lowered. According to an embodiment, the possible diagnoses are tuned based on epidemiology to improve the accuracy of the recommendations relative to the patient scenario of interest. Accordingly, the system can effectively recommend the diagnosis, and retrieve summarized test and treatment options from curated data sources for patient scenario. The overall model architecture, and the system components with flow charts are provided in the later slides.

At step 128 of the method, the ranked medical conditions and/or diagnosis are provided to the clinician. The medical conditions and/or diagnosis can be provided to the user via any user interface that allows information to be conveyed, such as a speaker or screen, among many other types of user interfaces. Alternatively, the medical conditions and/or diagnosis may be provided to a computing device or another automated system.

At optional step 126 of the method, the system generates a testing plan and/or treatment plan for the patient from the adjusted ranking of the at least one medical condition. For example, the system may determine or otherwise retrieve from memory a standard testing plan and/or treatment plan for the patient based on the highest-ranked diagnosis. Alternatively, the system may generate a de novo testing plan and/or treatment plan for the patient based on one or more identified diagnoses. For example, the system may recommend a test to distinguish between two possible diagnoses.

Accordingly, at step 130 of the method the system provides the generated testing plan and/or treatment plan for the patient to the clinician. The generated testing plan and/or treatment plan can be provided to the user via any user interface that allows information to be conveyed, such as a speaker or screen, among many other types of user interfaces. Alternatively, the medical conditions and/or diagnosis may be provided to a computing device or another automated system.

Referring to FIG. 3, in one embodiment, is a schematic representation of a system 300 or method for automated clinical diagnosis. At 312, the system receives a patient complaint, test result, or other clinician information, typically as natural language input. For example, a clinician may speak into a microphone, smartphone, or other user interface that receives natural language input. A natural language processing engine 314 receives the input and processes it to extract one or more patient symptoms 316 and one or more demographic parameters 318 for the patient, such as by identifying keywords, although other processes are possible.

At 320, the one or more extracted patient symptoms are weighted, such as based on specificity of their usage using log inverse frequency of their usage in a medical corpus, which may or may not be the same corpus utilized to create the knowledge graph. The weighted symptoms can now be queried on the knowledge graph 310.

At 322, an initial scattered forest is generated, with the start points being root nodes associated with the extracted and weighted one or more symptoms. According to an embodiment, one-hop expansions of the symptom nodes are made into knowledge graph 310 with the weights of the initial nodes as the activation weights.

At 324, the initial scattered forest is converted to a forest by adding context nodes. For example, according to an embodiment, the system expands the nodes which have minimum number of children to make it a connected graph from the forest. This expansion can be based on minimal context addition during the expansion. For example, the nodes can be expanded such that the system adds minimum number of nodes to make it a connected digraph from the forest. The expansion is stopped when there is a spanning tree structure.

An activation module 326 spreads the activation across the digraph. The activation is controlled using a sigmoid function and only partial activation flows to its children as inheritance of activation is proportional to number of siblings of the current node. Activation is a continuous process and it spreads from parent to children across the nodes.

A decay module 328 decays the activation. For example, as the activation spreads concurrently, the activation decays. Each time during the inheritance of activation the nodes lose a variable amount of activation. As the activation spreads, the nodes receive less activation if they are farther away from the base activation, due to the decay.

A control module 330 monitors the activation module 326 and the decay module 328, and stops the activation and decay cycle of the activations settle. The control module also ensures that there is no runaway activation among the nodes, and also controls the accumulation of activations at one node. The module stops the activation and decay cycle after the network stabilizes. The network always stabilizes as the activation weights are reduced at each inheritance.

At 332, the top ranked diseases and medical conditions are extracted from the knowledge graph. The signs and symptom information from online sources and curated medical sources are used to re-rank (refine) the list of diseases and medical conditions. At 334, the extracted demographic information 318 is utilized to fine-tune or otherwise adjust the ranking of diseases, diagnoses, and/or medical conditions. For example, if the disease is not common for the demographic, then its rank is lowered.

At 336, corresponding treatment and test information is extracted from the curated corpus and it is sent to a summarization module, where a summary of the treatment and test can be generated for the user.

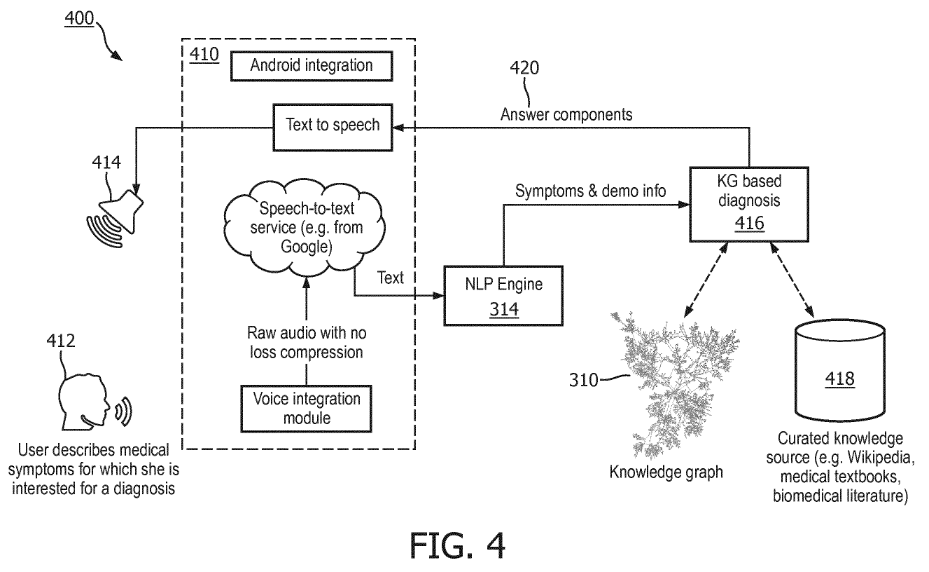

Referring to FIG. 4 is a schematic representation of a system 400 or method for automated clinical diagnosis. According to an embodiment, information is received via a natural language input from a patient or clinician, and the information is utilized to query a knowledge graph to generate a diagnosis. At module or system 410, information is received via a natural language input from a patient or clinician. According to an embodiment, the patient or clinician 412 speaks to a device or system comprising a microphone 414 or other device to detect the sound and convert it to digital signal. For example, module or system 410 may be a smartphone, recording device, or other device configured or capable of converting sound to a digital signal. For example, according to an embodiment, system 410 uses a speech-to-text service or module that converts sound to text.

System 410 generates text that is provided to a natural language processing engine 314, which processes the generated text to extract at least one patient symptom from the received information and at least one demographic parameter for the patient from the received information. For example, the natural language processing engine may extract keywords related to symptoms, such as lab results, procedures, and/or demographic information.

At 416, the extracted one or more patient symptoms and the extracted one or more pieces of demographic information are utilized to query the knowledge-graph 310 and provide one or more medical conditions, diagnoses, treatment plans, or testing plans. According to an embodiment, the knowledge-graph 310 is generated using information from a curated knowledge source 418.

At 420, one or more identified medical conditions, diagnoses, treatment plans, and/or testing plans are provided back to system 410. The information may be provided to the clinician or patient using any method. According to an embodiment, the information is converted to speech and is provided to the patient or clinician via a speaker 414, although many other methods for sharing the information are possible.

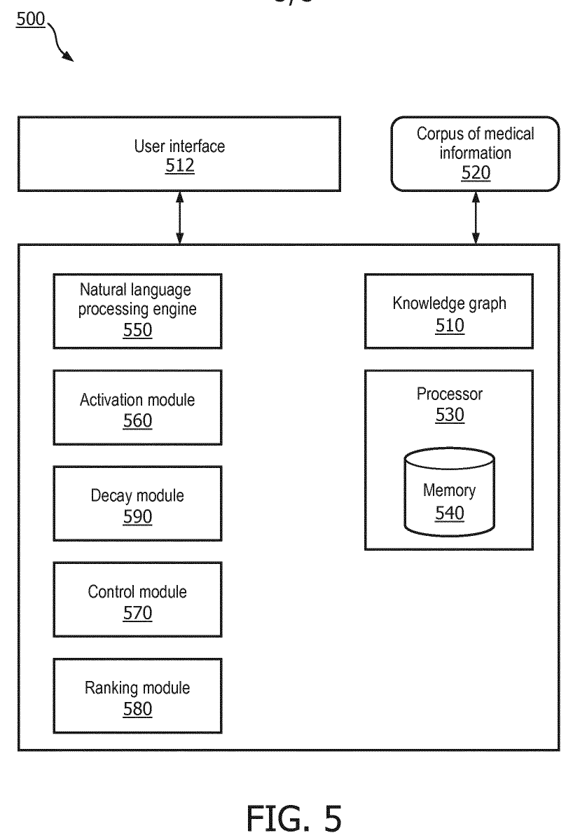

Referring to FIG. 5, in one embodiment, is a schematic representation of a system 500 for automated clinical diagnosis. System 500 can comprise any of the elements, engines, database, processors, and/or other components described or otherwise envisioned herein. According to an embodiment, system 500 comprises a knowledge graph 510 which is generated as described or otherwise envisioned herein from a corpus of medical information 520, which may be any source of information, including but not limited to medical journals, online news articles, online sources such as Wikipedia, and other sources.

According to an embodiment, system 500 comprises a processor which performs one or more steps of the method, and may comprise one or more of the engines or generators. Processor 530 may be formed of one or multiple modules, and can comprise, for example, a memory 540. Processor 530 may take any suitable form, including but not limited to a microcontroller, multiple microcontrollers, circuitry, a single processor, or plural processors. Memory 540 can take any suitable form, including a non-volatile memory and/or RAM. The non-volatile memory may include read only memory (ROM), a hard disk drive (HDD), or a solid state drive (SSD). The memory can store, among other things, an operating system. The RAM is used by the processor for the temporary storage of data. According to an embodiment, an operating system may contain code which, when executed by the processor, controls operation of one or more components of system 500.

According to an embodiment, system 500 comprises a user interface 512 to receive information from and/or provide information to a patient and/or clinician. The user interface can be any device or system that allows information to be conveyed and/or received, such as a speaker or screen, among many other types of user interfaces. The information may also be conveyed to and/or received from a computing device or an automated system. The user interface may be located with one or more other components of the system, or may located remote from the system and in communication via a wired and/or wireless communications network.

According to an embodiment, system 500 comprises a natural language processing engine 550 which processes the generated text to extract at least one patient symptom from the received information and at least one demographic parameter for the patient from the received information. For example, the natural language processing engine may extract keywords related to symptoms, such as lab results, procedures, and/or demographic information.

According to an embodiment, system 500 comprises an activation module 560 that spreads the activation across the digraph. The activation is controlled using a sigmoid function and only partial activation flows to its children as inheritance of activation is proportional to number of siblings of the current node. Activation is a continuous process and it spreads from parent to children across the nodes.

According to an embodiment, system 500 comprises a decay module 590 that decays the activation. For example, as the activation spreads concurrently, the activation decays. Each time during the inheritance of activation the nodes lose a variable amount of activation. As the activation spreads, the nodes receive less activation if they are farther away from the base activation, due to the decay.

According to an embodiment, system 500 comprises a control module 570 that monitors the activation module and the decay module, and stops the activation and decay cycle of the activations settle. The control module also ensures that there is no runaway activation among the nodes, and also controls the accumulation of activations at one node. The module stops the activation and decay cycle after the network stabilizes. The network always stabilizes as the activation weights are reduced at each inheritance.

According to an embodiment, system 500 comprises a ranking module 580 that ranks the one or more identified at least one medical condition and/or diagnosis based in part on information from one or more additional sources of medical information. For example, signs and symptom information from online sources and curated medical sources can be used to rank or refine the list of diseases and medical conditions. According to an embodiment, the ranking module 580 may fine-tune or adjust the ranking based on the extracted one or more demographic parameters.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of” “only one of,” or “exactly one of”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

1. A system for automated clinical diagnosis, the system comprising:
a knowledge graph generated from a corpus of medical information, the knowledge graph comprising a plurality of nodes, at least some of the nodes comprising a respective patient symptom and connected by an edge;
a user interface configured to receive natural language input from a user, the input comprising information about at least one patient symptom and at least one demographic parameter about the patient; and
a processor comprising a natural language processing engine configured to extract the at least one patient symptom and at least one demographic parameter from the received natural language input, wherein the processor is further configured to: (i) weight the extracted at least one patient symptom based at least in part on the frequency of the patient symptom in the corpus of medical information; (ii) query, using the weighted at least one patient symptom, the knowledge graph to generate a diagnosis graph as a subset of the knowledge graphs, querying the knowledge graph comprising processing the at least one patient symptom over multiple cycles across the medical knowledge graph, the diagnosis graph being a connected digraph representing the connected symptoms; (iii) identify a ranked list of one or more medical conditions, diagnoses, treatments, and/or tests for the patient from the diagnosis graph; and (iv) adjust, based on the extracted at least one demographic parameter about the patient, the ranking of the identified one or more medical conditions, diagnoses, treatments, and/or tests for the patient;
wherein the identified one or more medical conditions, diagnoses, treatments, and/or tests for the patient are provided to the user via the user interface.

2. The system of claim 1, wherein generating a diagnosis graph comprises the steps of: (i) assigning the assigned weight as an activation weight to a node of the knowledge graph; (ii) expanding the diagnosis graph to one or more connected nodes, wherein each expansion to a new connected node decays the activation weight; and (iii) concluding expansion when the activation weight is sufficiently decayed.

3. The system of claim 2, wherein the step of expanding the diagnosis graph to one or more connected nodes is repeated.

4. The system of claim 2, wherein the processor comprises a control module configured to monitor the expansion and decay of the diagnosis graph.

5. The system of claim 4, wherein the control module is further configured to stop expansion of the diagnosis graph when the diagnosis graph stabilizes.

6. The system of claim 1, wherein at least some of the edges of the knowledge graph are weighted.

7. The system of claim 1, wherein the highest ranked one or more medical conditions, diagnoses, treatments, and/or tests for the patient is provided to the user.

8. The system of claim 1, the processor is further configured to:
generate, from the adjusted ranking of one or more medical conditions for the patient, a testing plan and/or treatment plan for the patient; and
provide, to the clinician via the user interface, the generated testing plan and/or treatment plan for the patient.

9. The system of claim 1, wherein the extracted at least one patient symptom is weighted based on the log inverse frequency of the symptom in the corpus of medical information.

10. A method for automated clinical diagnosis, the method comprising the steps of:
providing an automated clinical diagnosis system comprising a knowledge graph generated from a corpus of medical information, the knowledge graph comprising a plurality of nodes, at least some of the nodes comprising a respective patient symptom and connected by an edge; a user interface configured to receive input from a user, the input comprising information about at least one patient symptom and at least one demographic parameter about the patient; and a processor;
receiving, via the user interface, information about a patient scenario, the information comprising at least one patient symptom and at least one demographic parameter for the patient;
extracting, using the processor, the at least one patient symptom from the received information;
extracting, using the processor, at least one demographic parameter for the patient from the received information;
weighting, using the processor, the extracted at least one patient symptom based at least in part on the frequency of the symptom in the curated corpus of medical information;
querying, using the weighted at least one patient symptom, the knowledge graph to generate a diagnosis graph as a subset of the knowledge graph querying the knowledge graph comprising processing the at least one patient symptom over multiple cycles across the medical knowledge graph, the diagnosis graph being a connected digraph representing the connected symptoms;
identifying a ranked list of one or more medical conditions, diagnoses, treatments, and/or tests for the patient from the diagnosis graph;
adjusting based on the extracted at least one demographic parameter about the patient, the ranking of the identified one or more medical conditions, diagnoses, treatments, and/or tests for the patient; and
providing the identified one or more medical conditions, diagnoses, treatments, and/or tests for the patient are provided to the user via the user interface.

11. The method of claim 10, wherein the processor comprises a natural language processing engine configured to extract the at least one patient symptom and at least one demographic parameter from the received input.

12. The method of claim 10, wherein the list of one or more medical conditions, diagnoses, treatments, and/or tests for the patient from the diagnosis graph is ranked based at least in part on information from one or more additional sources of medical information.

13. The method of claim 10, wherein the step of querying the knowledge graph to generate a diagnosis graph as a subset of the knowledge graph comprises the steps of:
assigning the assigned weight as an activation weight to a node of the knowledge graph;
expanding the diagnosis graph to one or more connected nodes, wherein each expansion to a new connected node decays the activation weight; and
concluding expansion when the activation weight is sufficiently decayed.

14. The method of claim 13, wherein the step of expanding the diagnosis graph to one or more connected nodes is repeated.

15. The method of claim 10, further comprising the step of generating the knowledge graph from the corpus of medical information.

16. The system of claim 1, wherein the processor is additionally configured to generate the knowledge graph from the corpus of medical information, generating the knowledge graph comprising adding a first node representing a first clinical concept, a second node representing a second clinical concept, and an edge between the first node and the second node according to a hierarchical relation of the corpus of medical information.

17. The system of claim 1, identifying a ranked list of one or more medical conditions, diagnoses, treatments, and/or tests for the patient from the diagnosis graph comprising retrieving candidate biometrical articles from the corpus of medical information, biometric articles from the corpus of medical information being indexed, the retrieving comprising searching through the indexed articles.
